(19) United States
(12) Patent Application Publication
(10) Pub. No.: US 2026/0102447 A1
(43) Pub. Date: Apr. 16, 2026

(54) APPLICATION OF YEAST SHELLS IN TREATMENT OF IMMUNE DISEASES RELATED TO MACROPHAGE IMMUNOMODULATION

(71) Applicant: The Second Affiliated Hospital of Xi'an Jiaotong University, Xi'an (CN)

(72) Inventors: Long ZHANG, Xi'an (CN); Li ZHANG, Xi'an (CN); Zhenxiong LIU, Xi'an (CN); Shuanying YANG, Xi'an (CN); Nan HU, Xi'an (CN)

(73) Assignee: The Second Affiliated Hospital of Xi'an Jiaotong University, Xi'an (CN)

(21) Appl. No.: 19/354,861

(22) Filed: Oct. 10, 2025

(30) Foreign Application Priority Data
Oct. 10, 2024 (CN) 2024114107120

(51) Int. Cl. A61K 36/06; A61K 9/00; A61K 9/16; A61P 19/02; A61P 37/06

(52) U.S. Cl. CPC A61K 36/06; A61K 9/0053; A61K 9/16; A61P 19/02; A61P 37/06

(57) ABSTRACT

An application of probiotic yeast shells in targeted immunomodulation drugs of inflammatory macrophages is provided, belonging to the field of biomedical technology. As a new type of drug, yeast shells have the characteristics of engineering, convenience, low cost, high patient adherence, safety, and excellent targeting. They are used as oral drugs for the treatment of chronic inflammatory diseases such as rheumatoid arthritis. They act on mitochondria via oxidative stress and oxidative phosphoric acid pathways to inhibit the overactivation of macrophages, reduce the release of inflammatory factors, and alleviate inflammatory responses. They can also promote the phagocytosis and antigen presentation of macrophages, enhance the body's immune defense ability, thereby regulating the immune function of macrophages. Yeast shells show great potential in the preparation of targeted immunomodulation drugs of inflammatory macrophages, providing a theoretical basis for the development of non-invasive, convenient, and safe treatment of chronic inflammatory diseases such as rheumatoid arthritis.

### CROSS-REFERENCE TO RELATED APPLICATIONS

The application claims priority to Chinese patent application No. 2024114107120, filed on Oct. 10, 2024, the entire contents of which are incorporated herein by reference.

### TECHNICAL FIELD

The present disclosure belongs to the field of drug immunomodulation technology, and specifically relates to an application of yeast shells in treatment of immune diseases related to macrophage immunomodulation.

### BACKGROUND

Rheumatoid arthritis (RA) is an autoimmune disease that seriously affects human life and health. More and more studies have shown that macrophages play an important role in the pathogenesis of RA. Therefore, the development of RA drugs targeting macrophages has become the focus of current research. However, current RA treatment options are limited by significant side effects, invasive administration methods, suboptimal targeting precision, and poor patient adherence. Therefore, developing a safe and effective oral drug that targets macrophages while improving patient adherence and convenience and avoiding trauma caused by parenteral administration (such as intravenous injection and intra-articular injection) remains a hot and difficult issue in the development of drugs against chronic inflammatory diseases such as RA.

Oral administration is currently the most widely used method of administration in clinical practices due to its safety, convenience, and better patient adherence. However, for some immunomodulatory drugs, traditional oral drugs are often difficult to specifically target macrophages after intestinal absorption, resulting in limited drug efficacy. Some immunomodulatory drugs have poor stability in the gastrointestinal environment and are easily degraded or inactivated, thereby reducing bioavailability. For patients with immune diseases who require long-term treatment, frequent injections or infusions not only increase the patient's pain and economic burden, but may also affect their quality of life. In addition, traditional oral drugs have limitations in the treatment of chronic inflammatory diseases such as RA.

The probiotic Saccharomyces cerevisiae (yeast) is not only widely used in food fields such as brewing and baking, but also used as a eukaryotic model organism in disease-related research. Studies have shown that β-glucan, the main component of yeast, is a safe product approved by U.S. Food and Drug Administration (FDA) to be beneficial to the human body. It can be targeted and recognized by Dectin-1 receptors on the surface of macrophages, such as hepatitis B virus vaccine, papillomavirus vaccine, and malaria vaccine, developed based on yeast expression hosts approved by FDA. These studies have shown that probiotic yeast has shown great potential and value in the field of immune regulation and vaccine development. The immunomodulatory function of yeast and its unique advantages as a vaccine development platform provide new ideas and methods for the prevention and treatment of various diseases. Studies have also found that in addition to being used for recombinant vaccine development, yeast can also be used in conjunction with the in vivo delivery of immune adjuvants and small molecule drugs, indicating that yeast has excellent oral properties.

Yeast shells, as a natural and degradable microvector, have good biocompatibility and stability. The applicant's previous research has proved that through different editing and transformation of yeast, the modified yeast shells can coordinate the oral delivery of editable drugs such as RNA and recombinant yeast vectors (yeast-gene vectors). The RNA drugs delivered by yeast shells as vectors and recombinant yeast vectors ameliorate the inflammatory response of osteoarthritis and RA via the gut-joint axis immunity, indicating that yeast shells can not only effectively protect the drugs carried from damage by the gastrointestinal environment, but also accurately deliver drugs to the lesion through a specific immune pathway to achieve the effect of local treatment, showing good versatility.

Although this immunomodulation mediated by yeast shells as a vector has shown safety, immunogenicity, oral availability, convenience, and non-invasiveness, it is still unclear whether yeast shells can be directly used as an oral drug to target the molecular mechanism of macrophage immunomodulation, whether it is dose-dependent, and how yeast shells participate in the RA treatment via the gut-joint axis immunity. In view of the complexity of RA, traditional oral drugs have limitations in the treatment of chronic inflammatory diseases such as RA. Therefore, exploring strategies for multi-target combination therapy that simultaneously inhibit multiple pro-inflammatory signaling pathways or promote both anti-inflammatory and repair mechanisms is an important approach to achieving more comprehensive RA treatment.

### SUMMARY

In view of the problems of low bioavailability, invasive administration methods, suboptimal targeting precision, and poor patient adherence of existing RA treatment drugs, it is urgent to develop a safe and effective oral targeted macrophage immunomodulatory drug for treating RA. There are no patents or literature reports on the technical status of using yeast shells alone as oral drugs instead of vectors for preparing drugs in treatment of immune diseases related to macrophage immunomodulation. The present disclosure aims to provide an application of yeast shells in treatment of immune diseases related to macrophage immunomodulation.

To achieve the preceding purpose, the present disclosure provides the following technical solutions:

The present disclosure provides a new application of yeast shells in the preparation of drugs for treating immune diseases related to macrophage immunomodulation, in which the yeast shells are directly used as drugs, the drugs are oral drugs, and the yeast shells are derived from yeast.

The dosage form of the drug is selected from a group consisting of tablet, pill, powder, suspension, gel, emulsion, cream, granule, nanoparticle, capsule, suppository, injection, spray, and ampoule.

The drug is a drug for preventing and/or treating immune diseases related to macrophage immunomodulation.

The drug is a drug for preventing and/or treating RA.

RA includes clinically early, established or advanced RA.

The symptoms of RA include any one or more of humoral immune imbalance, imbalance between the body's oxidation and antioxidant capacity, increased inflammatory cytokines, joint swelling, joint stiffness, joint pain, bone injury, bone deformity, rheumatoid nodules and articular cartilage destruction.

The present disclosure provides an oral drug for preventing and/or treating immune diseases related to macrophage immunomodulation, and the oral drug includes yeast shells. The oral drug further includes one or more pharmaceutically acceptable excipients.

The pharmaceutically acceptable excipient is at least one selected from a group consisting of pharmaceutically acceptable solvents, solubilizers, cosolvents, adhesives, emulsifiers, colorants, disintegrants, fillers, lubricants, wetting agents, osmotic pressure regulators, stabilizers, glidants, preservatives, flavoring agents, suspending agents, coating materials, aromatics, anti-adhesives, pH regulators, integrators, penetration enhancers, buffers, plasticizers, surfactants, moisturizers, thickeners, inclusion agents, absorbents, release blockers, diluents, flocculants and deflocculants, filter aids, polymer framework materials and film-forming materials.

The present disclosure provides a method for preparing the preceding oral drug for preventing and/or treating immune diseases related to macrophage immunomodulation, including preparing yeast shells together with pharmaceutically acceptable excipients. The method for preparing the yeast shells is as follows: dissolving yeast in water, adjusting the pH value to 11-13 with alkali and then performing alkali treatment; centrifuging, resuspending the resulting precipitate in water, adjusting the pH value to 3-5 with acid and then performing acid treatment; centrifuging, collecting and washing the precipitate, and air-drying it at room temperature to obtain yeast shells.

The alkali treatment is performed at 50-70° C. for 1-2 h.

Further, the alkali is selected from any one of NaOH, sodium carbonate, and sodium bicarbonate.

The acid treatment is performed at 50-60° C. for 1-2 h.

Further, the acid is selected from any one of HCl, acetic acid, and carbonic acid.

The solvents used in sequence for cleaning the precipitate are deionized water, anhydrous isopropanol, and acetone.

Compared with the prior art, the present disclosure achieves the following technical effects:

The application of yeast shells provided by the present disclosure in the preparation of drugs for treating immune diseases related to macrophage immunomodulation creates a new treatment strategy by directly using yeast shells as the drug instead of traditional drug vectors. This direct application method reduces the complexity of preparation and potential adverse reactions, and makes treatment simpler and safer. Traditionally, yeast shells are mainly used to carry and deliver drugs, but the present disclosure proposes for the first time to use them directly as drugs. This direct application method avoids the complex drug loading process, reduces preparation steps, and potential risks; by directly using yeast shells as oral drugs, biologically active ingredients such as β-glucan on the surface of yeast shells can specifically bind to Dectin-1 receptors on the surface of macrophages. This binding mechanism enables yeast shells to directly target macrophages after oral administration, thereby significantly improving the targeting of drugs, helping reduce the distribution of drugs in non-target tissues, reduce side effects, and improve therapeutic effects. The new application provided by the present disclosure not only simplifies the drug preparation process and reduces potential adverse reactions, but also significantly improves the simplicity and safety of treatment, creating a new treatment strategy.

Further, the present disclosure provides a new route of administration and dosage form selection for immunomodulatory drugs by using yeast shells as oral drugs, which helps expand the application range of immunomodulatory drugs, especially for patients with immune diseases who require long-term treatment or are difficult to administer through other routes.

Further, the application of the present disclosure directly targets macrophages and regulates their immune function. Yeast shells can more effectively inhibit inflammatory responses and reduce the release of inflammatory factors, thereby alleviating disease symptoms and reducing recurrence. The direct immunomodulatory effect is more accurate and effective than traditional drugs.

The oral drugs provided by the present disclosure can directly enter the body and act on macrophages, act on mitochondria via oxidative stress and oxidative phosphoric acid pathways, inhibit the overactivation of macrophages, regulate their immune function to inhibit inflammatory response, reduce the release of inflammatory factors, and alleviate inflammatory response. This direct immunomodulatory effect helps ameliorate disease symptoms and reduce disease recurrence. Compared with traditional drugs, yeast shells, as natural products, are safer and have fewer side effects. In addition, since yeast shells have good biocompatibility and degradability, they will not cause serious rejection reactions or residual problems in the body, reducing the side effects that may be caused by traditional drugs for treating RA, such as gastrointestinal discomfort and immune system suppression. Yeast shells show great potential in preparing drugs for treating immune diseases related to macrophage immunomodulation.

The method for preparing a drug containing yeast shells provided by the present disclosure has clear steps and simple operation, can efficiently obtain high-quality yeast shells, has high repeatability, is suitable for industrial production, and has broad application prospects and market value in the preparation of drugs for treating immune diseases related to macrophage immunomodulation.

### BRIEF DESCRIPTION OF DRAWINGS

FIG. 1 shows the yeast shells of the present disclosure participating in the regulation of macrophage oxidative stress and oxidative phosphorylation functions, in which A is a cluster analysis of differentially expressed genes between the YS treatment group and the LPS-Mφ control group; B is a comparison of differentially expressed genes between the YS treatment group and the LPS-Mφ control group; and C is a bubble chart enrichment statistics between the LPS-Mφ control group and the YS treatment group.

In FIG. 2, A is a GO analysis of the YS treatment group and the LPS-Mφ control group; and B is a correlation analysis between the YS treatment group and the LPS-Mφ control group.

FIG. 3 shows the results of GSEA bioinformatic analysis of differentially expressed genes in the LPS-Mφ control group and the YS treatment group.

FIG. 4 shows the results of ROS expression in macrophages induced by different doses of YS.

FIG. 5 shows the results of down-regulation of mitochondrial membrane potential and apoptosis in RAW264.7 macrophages induced by different doses of YS, in which A is the staining result of mitochondrial membrane potential and apoptosis, B is the statistics of mitochondrial membrane potential Mito-tracker and apoptosis Annexin V after co-culture of YS with RAW264.7 macrophages for 48 h, and C is the statistics of mitochondrial membrane potential Mito-tracker and apoptosis Annexin V after co-culture of YS with M1 macrophages for 72 h.

FIG. 6 shows the results of down-regulation of ATP expression in RAW264.7 macrophages induced by different doses of YS. The results were not significant (n.s.), ***P<0.001, and n=3.

FIG. 7 shows the results of different doses of YS inducing macrophages to produce pyroptosis-related proteins, in which A is a molecular mechanism diagram of YS-induced pyroptosis of RAW264.7 macrophages, B is the detection of pyroptosis-related proteins of RAW264.7 macrophages induced by different doses of YS, and C is the quantitative analysis of pyroptosis protein WB detection; and each protein expression analysis was compared with the control group (ctrl) and calibrated with the ctrl of each protein indicator. The results were not significant (n.s.), *P<0.05, **P<0.01, ***P<0.001, and n=3.

FIG. 8 shows that probiotic yeast shells can regulate the microecology of intestinal flora after oral administration, in which A is the effect of YS on Drug Class, Resistance Mechanism, and Gene Family in intestinal flora, B is the effect of YS on the classification of intestinal flora, and C is the effect of YS on amino acid metabolism, protein synthesis, and DNA replication of flora (n=8).

FIG. 9 shows the amelioration of RA symptoms after oral administration of probiotic yeast shells, in which a is the construction process of the RA mouse model, b is the standard chart of RA mouse joint scoring, c is the chart of RA mouse joint symptoms after different treatments, d is the statistical results of the scores of RA mice in different treatment groups, e is the relative cure effect of RA mice after different treatments, and f is the weight change of mice from day 1 to day 44, g is the weight change of RA mice before and after treatment, h is the weight statistics of the main organs of mice. The results were not significant (n.s.), *P<0.05, **P<0.01, ***P<0.001, and n=7-8.

FIG. 10 shows the results of safranin-fixing green staining and joint scoring of the ankle joint, articulatio cuneonavicularis, and metatarsocuneiform joint of a mouse paw, in which A is the result of safranin-fixing green staining of the ankle joint, B is the result of safranin-fixing green staining of the articulatio cuneonavicularis, C is the result of safranin-fixing green staining of the metatarsocuneiform joint, and D is the score of the ankle joint, articulatio cuneonavicularis, and metatarsocuneiform joint of the mouse paw. The results were not significant (n.s.), *P<0.05, **P<0.01, ***P<0.001, and n=6-7.

FIG. 11 shows the histological toxicity of YS evaluated by HE staining of major tissues and organs in the heart, liver, lung, and kidney of mice.

### DETAILED DESCRIPTION OF THE EMBODIMENTS

In order to enable those skilled in the art to better understand solutions of the present disclosure, the following will combine the accompanying drawings in the embodiments of the present disclosure to clearly and completely describe the technical solutions in the embodiments of the present disclosure. Obviously, the described embodiments are some but not all embodiments of the present disclosure. Based on the embodiments described in the present disclosure, all other embodiments obtained by those of ordinary skill in the art without creative work are within the scope of protection of the present disclosure.

If the specific experimental steps or conditions are not specified in the embodiment, it can be carried out according to the operation or conditions of the conventional experimental steps described in the literature in this field. The reagents or instruments used without indicating the manufacturer are all conventional reagent products that can be obtained through commercial purchase.

The macrophage RAW264.7 used in the present disclosure was purchased from BeNa Culture Collection.

DMEM medium: a high-glucose medium containing 4.5 g/L D-glucose, 4.5 g/L L-glutamine, and 110 mg/L sodium pyruvate.

### Embodiment 1

This embodiment provided the preparation of engineered yeast shells, that is, preparing engineered yeast shell (YS) by acid-alkali treatment and extraction by chemical reagent. Total 20 g of yeast was dissolved in 100 mL of deionized water to obtain a yeast aqueous solution; the pH value of yeast aqueous solution was adjusted to 12.5 with 1 M NaOH solution, the solution was treated at 60° C. for 1 h, centrifuged for 5 min, and the supernatant was discarded; the yeast was resuspended with 100 mL of sterile deionized water and mixed well, the pH was adjusted to 4 with concentrated HCl solution, the solution was treated at 55° C. for 1 h, centrifuged for 5 min, and the precipitate was collected; the precipitate was washed 2 times with sterile deionized water, 4 times with anhydrous isopropanol, and 2 times with acetone; the precipitate was then placed in a glass dish and air dried at room temperature to obtain the engineered yeast shell, or YS.

### Embodiment 2

Immunogenicity test of yeast shell (YS): YS (10⁶/6-well plate cells) prepared in Embodiment 1 was co-cultured with RAW264.7 macrophages (Mφ) induced by lipopolysaccharide (LPS) (100 ng/mL) for 24 h (YS treatment group), and the cells were collected after 24 h of culture for RNA-seq sequencing. At the same time, RAW264.7 macrophages induced by LPS were used as the LPS-Mφ control group (NC group). The sequencing results were analyzed by Gene Ontology (GO), Kyoto Encyclopedia of Genes and Genomes (KEGG), and Gene Set Enrichment Analysis (GSEA) to explore how YS is involved in the regulation of macrophage biological functions.

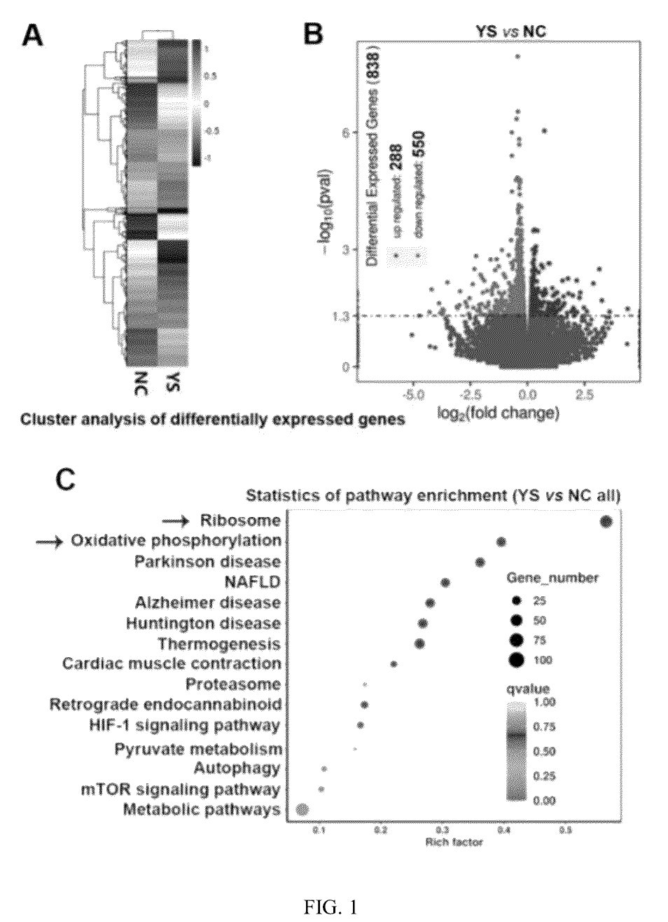

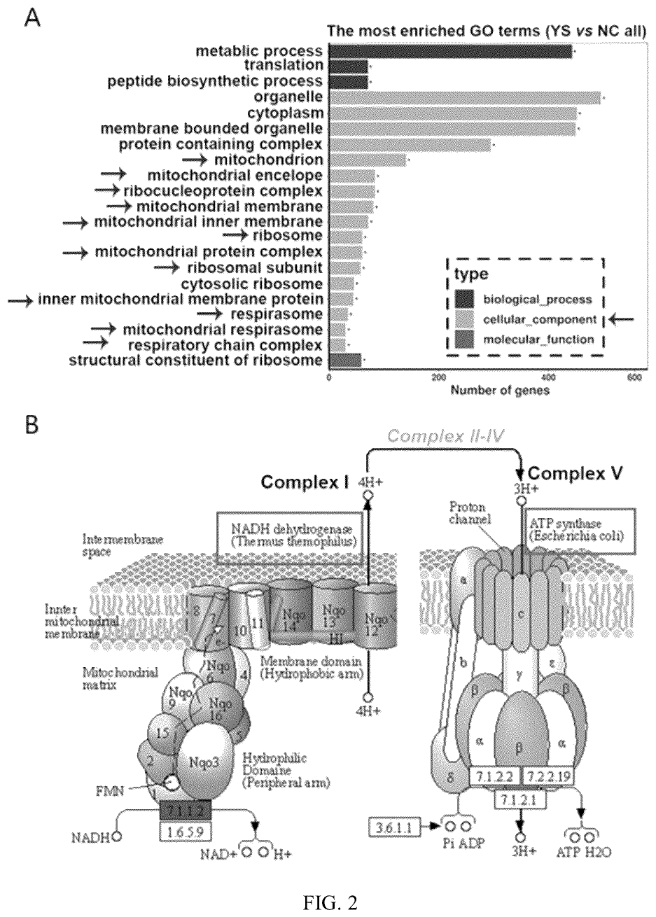

In order to explore the immunomodulatory mechanism of YS on macrophages, YS was co-incubated with RAW264.7 macrophages induced by LPS. After 24 h, the cells were collected for RNA-seq high-throughput sequencing and GSEA bioinformatic analysis. The specific results are shown in FIGS. 1-3.

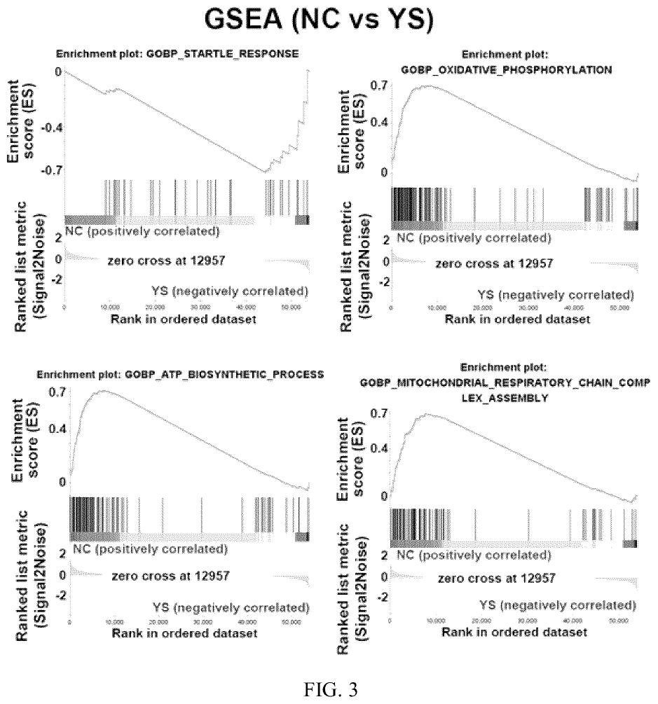

In FIG. 1, A is a cluster analysis of differentially expressed genes between the YS treatment group and the LPS-Mφ control group; B is a comparison of differentially expressed genes between the YS treatment group and the LPS-Mφ control group; and C is a bubble chart enrichment statistics between the LPS-Mφ control group and the YS treatment group. In FIG. 2, A is a GO analysis of the YS treatment group and the YS treatment group; and B is a correlation analysis of the YS treatment group and the LPS-Mφ control group. FIG. 3 shows the results of GSEA bioinformatic analysis of differentially expressed genes in the LPS-Mφ control group and the YS treatment group.

After YS and LPS-Mφ (LPS-induced RAW264.7 macrophages) were co-cultured, compared with the LPS-Mφ control group, YS treatment significantly changed the gene expression pattern of macrophages, indicating that YS has an important effect on the biological function of macrophages. Compared with the LPS-Mφ control group (NC group), YS treatment significantly up-regulated 288 genes and down-regulated 550 genes, indicating that YS has a regulatory effect on gene expression in macrophages, involving multiple biological processes and pathways. Compared with the LPS-Mφ control group (NC group), differentially expressed genes were mainly enriched in ribosome and oxidative phosphorylation-related pathways, which are closely related to energy metabolism and protein synthesis, indicating that YS regulates the immune function of macrophages by affecting these processes. The GO analysis results of the YS-treated group and the NC group show that differential genes were associated with mitochondria, further supporting that YS regulates macrophage immune function by affecting mitochondrial function. The results of KEGG Pathway analysis of differentially expressed gene enrichment show that YS treatment significantly affected the expression of genes related to oxidative phosphorylation, which play a key role in energy metabolism and ATP synthesis, further proving the mechanism by which YS affects macrophage function through the oxidative phosphorylation pathway. GSEA bioinformatics analysis shows that YS is involved in regulating macrophage stress response, oxidative phosphorylation, ATP biosynthesis process, mitochondrial respiratory chain complex, and so on, further confirming that YS acts on mitochondria through oxidative stress and oxidative phosphorylation pathways, thereby affecting the immunomodulatory function of macrophages.

In summary, RNA-seq and GSEA analysis revealed the complex role of YS in the immunomodulatory mechanism of macrophages. YS affects the immune function of macrophages by broadly regulating gene expression, especially genes related to ribosomes, oxidative phosphorylation, and mitochondrial function, which not only helps understand the mechanism of action of YS, but also provides clues for the development of new immunomodulatory strategies.

### Embodiment 3

The results of RNA-seq high-throughput sequencing and GSEA analysis (oxidative stress) in Embodiment 2 show that YS may stimulate macrophage Mφ to produce oxidative stress, induce ROS reactive oxygen generation and oxidative phosphorylation, thereby leading to mitochondrial function damage and inducing apoptosis in inflammatory Mφ.

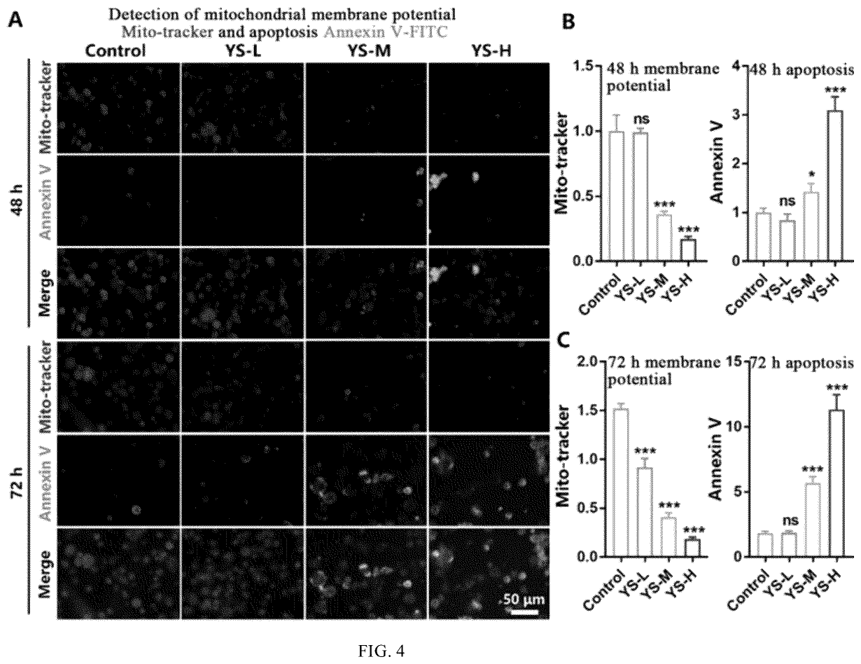

RAW264.7 macrophages were cultured in 6-well plates and induced with 100 ng/mL LPS for 24 h. Different doses of YS (YS-L group (low-dose group): 0.5*10⁶/6-well plate cells, YS-M group (medium-dose group): 10⁶/6-well plate cells, YS-H group (high-dose group): 2*10⁶/6-well plate cells) were added to the cell culture medium and co-cultured with the macrophages for 48 h and 72 h, respectively. Total 10 μL of active oxygen ROS fluorescent probe (DCFH-DA fluorescent probe) was added to 10 mL DMEM culture medium and mixed well, and then 500 μL of fluorescent probe mixture was added to the cells for treatment at room temperature in the dark for 30 min. The cells were washed 3 times with 1 mL of PBS and then observed under a fluorescence microscope. The specific results are shown in FIG. 4. At the same time, RAW264.7 macrophages induced by LPS were used as the LPS-Mφ control group (NC group).

It can be seen from the data in FIG. 4 that after 48 h of co-culture: Compared with the control group, as the YS dose increased, Mφ was induced to produce more ROS, indicating that YS can induce oxidative stress at lower concentrations, and the effect increases with increasing concentration. Co-culture for 72 h: With the continuous consumption of nutrients in the culture medium, ROS expression in all YS-treated groups increased significantly, reflecting that the extension of culture time exacerbated the stress state of cells, and a large number of cell apoptosis was observed under the microscope, further confirming that YS caused cell damage and apoptosis by inducing ROS production. The high-dose YS-H group produced more ROS than other groups (including the control group, low-dose group and medium-dose group), which again shows that YS dose-dependently induces macrophage oxidative stress and expresses ROS.

### Embodiment 4

In this embodiment, the mitochondrial membrane potential-dependent red fluorescent probe Mito-Tracker Red and the apoptosis green fluorescent probe Annexin V-FITC were used to detect the effects of different doses of YS on macrophage mitochondrial membrane potential and apoptosis.

RAW264.7 macrophages were cultured in 6-well plates. After LPS (100 ng/mL) was used to induce RAW264.7 macrophages for 24 h, different doses of YS (YS-L group: 0.5*10⁶/6-well plate cells, YS-M group: 10⁶/6-well plate cells, YS-H group: 2*10⁶/6-well plate cells) were added to the cell culture medium and co-cultured with the macrophages for 48 h and 72 h, respectively; the cell culture fluid was removed and added with PBS to wash once; 188 μL of Annexin V-FITC binding solution and 5 μL of Annexin V-FITC were added and mixed gently; then 2 μL of Mito-Tracker Red CMXRos staining solution and 5 μL of Hoechst 33342 staining solution were added and mixed gently; the mixture was incubated at room temperature (20° C.-25° C.) for 30 min in the dark, and then placed in an ice bath; immediately after observation under a fluorescence microscope, Mito-Tracker Red CMXRos showed red fluorescence, Annexin V-FITC showed green fluorescence, and Hoechst 33342 showed blue fluorescence. The test results are shown in FIG. 3.

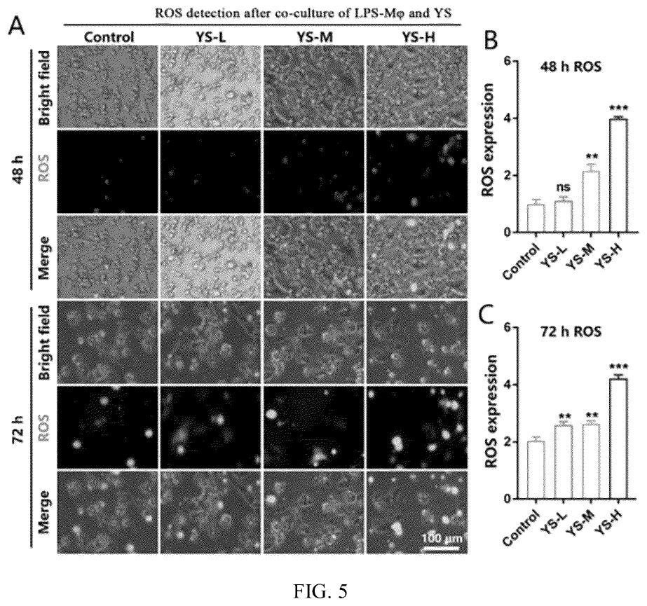

In FIG. 5, A is the staining result of mitochondrial membrane potential and apoptosis, B is the statistics of mitochondrial membrane potential Mito-tracker and apoptosis Annexin V after co-culture of YS with RAW264.7 macrophages for 48 h, and C is the statistics of mitochondrial membrane potential Mito-tracker and apoptosis Annexin V after co-culture of YS with RAW264.7 macrophages for 72 h.

The results of mitochondrial membrane potential and apoptosis staining show that: With the increase of YS dose (from YS-L group to YS-H group), red fluorescence (representing mitochondrial membrane potential) gradually weakened, especially in the YS-H group, the intensity of red fluorescence decreased significantly, indicating that the mitochondrial membrane potential was down-regulated. At the same time, green fluorescence (representing cell apoptosis) increased significantly in the YS-H group, indicating that high-dose YS treatment induced macrophage apoptosis.

The statistics of 48 h co-culture results show that: With the increase of YS concentration, the value of mitochondrial membrane potential (Mito-tracker) gradually decreased, and there was a significant difference between the YS-H group and the control group (***P<0.001), indicating that high doses of YS significantly down-regulated mitochondrial membrane potential; the value of apoptosis (Annexin V) significantly increased in the YS-H group, further confirming that high doses of YS induced macrophage apoptosis.

The statistics of the 72-h co-culture results show that after 72 h of co-culture, the mitochondrial membrane potential of macrophages in the YS-H group further decreased rapidly, indicating that mitochondrial function damage was aggravated. At the same time, more RAW264.7 macrophages underwent apoptosis and Annexin V values increased significantly.

In summary, YS can induce the down-regulation of mitochondrial membrane potential of RAW264.7 macrophages in a dose-dependent manner, indicating that YS has a damaging effect on mitochondrial function; high-dose treatment with YS significantly promoted macrophage apoptosis, which is related to the down-regulation of mitochondrial membrane potential and the increase of oxidative stress. These results show that YS induces apoptosis of inflammatory macrophages by affecting mitochondrial function, and YS can cause damage to the mitochondrial function of inflammatory macrophages.

### Embodiment 5

The decrease in mitochondrial membrane potential in Embodiment 4 indicates that mitochondrial function is impaired, which in turn affects the expression of adenosine triphosphate (ATP). In this embodiment, different doses of YS were co-cultured with LPS-Mφ cells for 48 h and 72 h, and then transfected into pCMV-Mito-ATP fluorescent probe. Based on the mitochondrial localization signal (MLS) AT1.03 fluorescent protein carried by pCMV-Mito-ATP fluorescent probe, Mitochondrial ATP expression was observed by fluorescence microscopy.

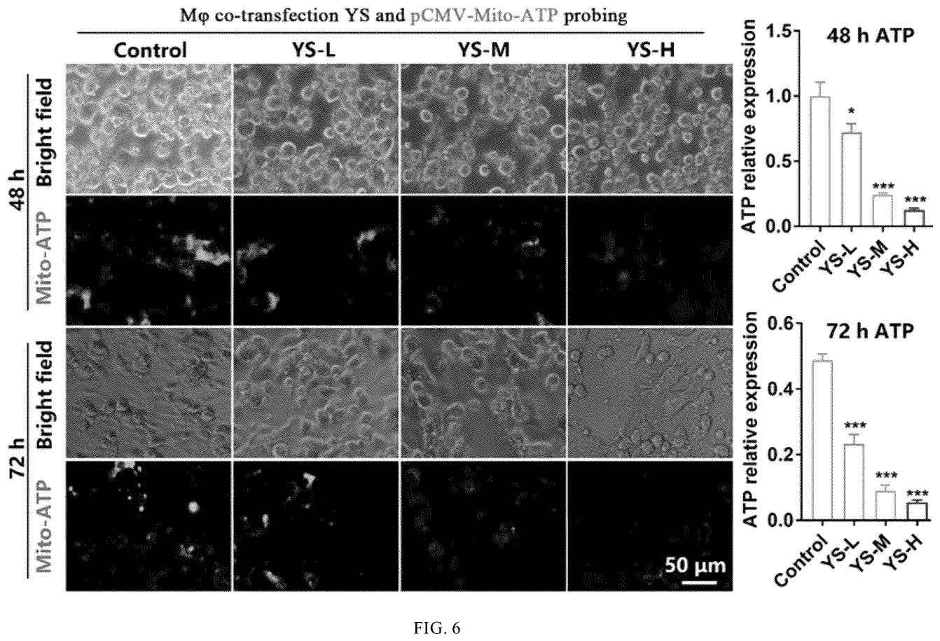

After inducing RAW264.7 macrophages with LPS (100 ng/ml) for 24 h, 5 μg of pCMV-Mito-ATP fluorescent probe was transfected into the macrophages using Lipofectamine™ 3000 transfection reagent. After 12 h of transfection, different doses of YS (YS-L group: 0.5*10⁶/6-well plate cells, YS-M group: 10⁶/6-well plate cells, YS-H group: 2*10⁶/6-well plate cells) were added to the cell culture medium and co-cultured with macrophages. After 48 h and 72 h, the mitochondrial ATP expression was observed by fluorescence microscopy. The specific results are shown in FIG. 6.

As can be seen from the data in FIG. 6, all doses of YS inhibited ATP production to a certain extent compared with the control group (Control). This inhibitory effect becomes more significant when the YS dose increases, indicating that the inhibitory effect of YS on ATP expression tends to be negatively correlated with its dose. At the two time points of 48 h and 72 h, the inhibitory effect of YS on ATP expression was more obvious at 72 h, and the long-term impact of YS on mitochondrial function was more serious, thereby further reducing the production of ATP. The high-dose YS-H group significantly inhibited the production of ATP at both 48 h and 72 h (*P<0.001), indicating that at higher concentrations, YS has a stronger inhibitory effect on mitochondrial ATP synthesis. As the YS dose increases, the green fluorescence gradually weakens, which intuitively reflects the decrease in ATP expression levels.

In summary, YS can significantly inhibit the production of ATP in inflammatory macrophages, and the expression level of ATP is negatively correlated with the dose of YS. Compared with the control group and the low-dose YS-L group, the high-dose YS-H group significantly inhibited the production of ATP.

### Embodiment 6

The above embodiment tested that YS-induced macrophage death may be related to YS-mediated mitochondrial function damage and thus induce RAW264.7 macrophage death. Therefore, the present disclosure speculates that YS can induce pyroptosis (inflammatory macrophage death) of RAW264.7 inflammatory macrophages. In order to verify this hypothesis, in the present disclosure different doses of YS were co-cultured with LPS-induced M1 macrophages and cellular proteins were collected. The expression of pyroptosis-related proteins NLRP3, Caspase-1, GSDMD-n, IL-1B, and IL-18 was detected by Western Blot.

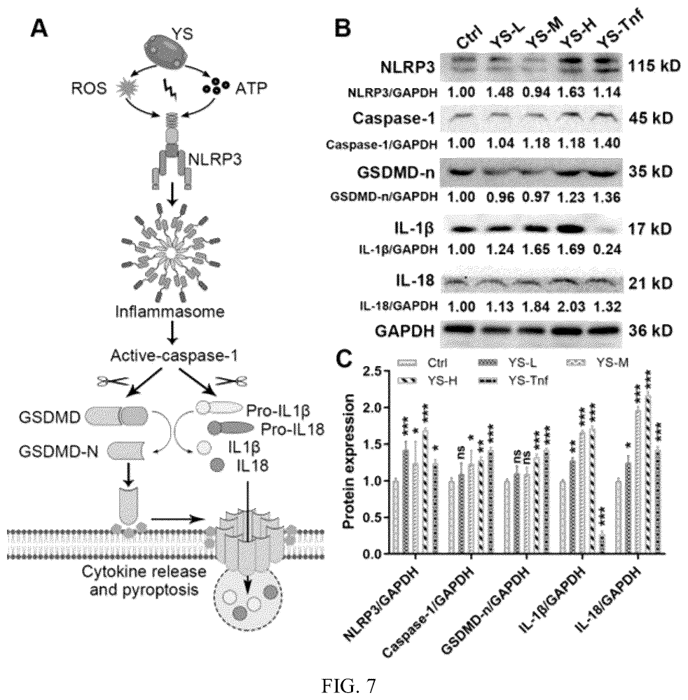

RAW264.7 macrophages were cultured in 6-well plates and induced with LPS (100 ng/ml) for 24 h. Then different doses of YS (YS-L group: 0.5*10⁶/6-well plate cells, YS-M group: 10⁶/6-well plate cells, YS-H group: 2*10⁶/6-well plate cells) were added to the cell culture medium and co-cultured with the macrophages for 48 h before collecting cellular proteins. The expression of pyroptosis-related proteins NLRP3, Caspase-1, GSDMD-n, IL-1B, and IL-18 was detected by Western Blot. The specific test results are shown in FIG. 7.

In FIG. 7, A is the molecular mechanism diagram of YS-induced pyroptosis of RAW264.7 macrophages, B is the detection of pyroptosis-related proteins of RAW264.7 macrophages induced by different doses of YS, and C is the quantitative analysis result of pyroptosis protein WB detection.

Molecular mechanism of YS-induced pyroptosis of RAW264.7 macrophages: YS induces the production of ROS by acting on mitochondria, thereby activating NLRP3 inflammasome. The activated NLRP3 recruits and activates Caspase-1, prompting GSDMD (Gasdermin D) protein to be cut into GSDMD-N fragments, which are then inserted into the cell membrane to form pores. It causes cells to swell and release inflammatory factors such as IL-1ß and IL-18, eventually causing pyroptosis.

Detection of pyroptosis-related proteins of RAW264.7 macrophages induced by different doses of YS: With the increase of YS dose, the expression level of each protein also shows an upward trend, especially the increase in GSDMD-n and IL-1B/IL-18 expression is more significant, indicating that the YS-induced pyroptosis process is activated and is closely related to the dose of YS.

Quantitative analysis of pyroptosis protein WB detection: The expressions of NLRP3 in the YS treatment groups (YS-L group, YS-M group, and YS-H group) were significantly increased compared with the control group, and they increased with the increase of YS dose, but the difference between the groups did not reach a statistically significant level. The expression of Caspase-1 also showed an increasing trend with the increase of YS dose, but there was no significant difference in the YS-L group, while it increased significantly in the YS-M and YS-H groups (*P<0.05, P<0.01). As a key executive protein of pyroptosis, the expression of GSDMD-n increased significantly in the YS treatment group and increased significantly with the increase of YS dose (*P<0.001). The YS treatment group significantly promoted the release of IL-1ß and IL-18, which increased significantly with increasing YS doses (*P<0.001).

The above data show that YS-induced inflammatory macrophage death actually works through the pyroptosis pathway, that is, YS can play an immunomodulatory role through the mitochondrial-pyroptosis pathway.

### Embodiment 7

### (1) Construction of RA Model Mice and Dosage Regimen

Total 2 mg/mL bovine collagen and complete Freund's adjuvant (CFA) (4 mg/mL Mycobacterium tuberculosis) were mixed in a volume of 1:1 and stirred with a homogenizer on ice to obtain an emulsion. The emulsion prepared using type II collagen and complete Freund's adjuvant was injected subcutaneously at the root of the mouse tail to create an RA model for 8-week-old DBA/1 mice. Day 1: Mice were subcutaneously injected with mycobacterium type II collagen containing CFA (1 mg/mL) at the base of their tails; Day 23: Subcutaneous booster injection, with type II collagen emulsion containing CFA (5 mg/mL mycobacteria) injected. The successfully modeled RA mice were randomly divided into 5 groups: PBS group, YS-L group (low-dose group), YS-M group (medium-dose group), YS-H group (high-dose group) and YS-PG group (positive control group). See Table 1 for details. The initial body weight, daily body weight, and joint RA score (according to Chondrex, Inc) of the mice were recorded during the experiment.

TABLE 1
Animal Treatment and Grouping

| | | Low- | Medium- | High- | Positive |
|---|---|---|---|---|---|
| | Control | dose | dose | dose | Control |
| | Group | Group | Group | Group | Group |
| | (n = 8) | (n = 8) | (n = 8) | (n = 8) | (n = 8) |
| Route of | PBS | YS-L | YS-M | YS-H | YS-PG |
| Administration | | | | | |
| in RA Mice | | | | | |

Drug induction was performed on 8-week-old male DBA/1 mice using a booster immunization regimen of subcutaneous injection at the base of the mouse tail. The steps were as follows: Day 1: Subcutaneous injection of mycobacterium type II collagen containing CFA (1 mg/mL); Day 15/25/30/34/36/39/41/43: the mouse joints were scored respectively; Day 23: Subcutaneous booster injection of type II collagen emulsion containing CFA (5 mg/ml mycobacteria); Day 10-44: YS treatment was given every 3 days.

During the continuous administration of mice, each group of mice was treated as follows: \textcircled{1} Each mouse in the PBS group was given 100 μL of PBS by oral gavage every 3 days; \textcircled{2} Each mouse in the YS-L group was gavaged with 100 μL of YS-L solution (containing 0.5*10⁶ YS); \textcircled{3} Each mouse in the YS-M group was gavaged with 100 μL of YS-M solution (containing 10⁶ YS); 4̅ Each mouse in the YS-H group was gavaged with 100 μL of YS-H solution (containing 2*10⁶ YS); \textcircled{5} Each mouse in the YS-PG group was gavaged with 100 μL of YS-PG solution (containing 10⁶ YS and 4 mg PG).

### (2) Anatomical and Histological Evaluation of YS Safety

After RA mice were treated with different doses of YS, on Day 44, \textcircled{1} the heart, liver, spleen, lung, and kidney were collected and their weights were measured to evaluate whether YS had an effect on major organ weights (organ damage) from anatomical perspective; \textcircled{ } 2 HE staining was performed on the heart, liver, lung, kidney, and intestine to evaluate the histological toxicity of YS at the histological level.

### (3) Metagenomics Evaluation of the Effect of YS on Intestinal Flora Microorganisms

After RA mice were treated with YS, intestinal contents were collected for metagenomic sequencing (entrusted to Beijing Allwegene Technology Co., Ltd.). The flora composition of the intestinal microbiome was analyzed by sample composition and functions to evaluate the regulatory effect of YS on the development of RA disease. Moreover, whether YS affects the microbial diversity of intestinal flora was studies and the biosafety of YS was evaluated at the metagenomic level.

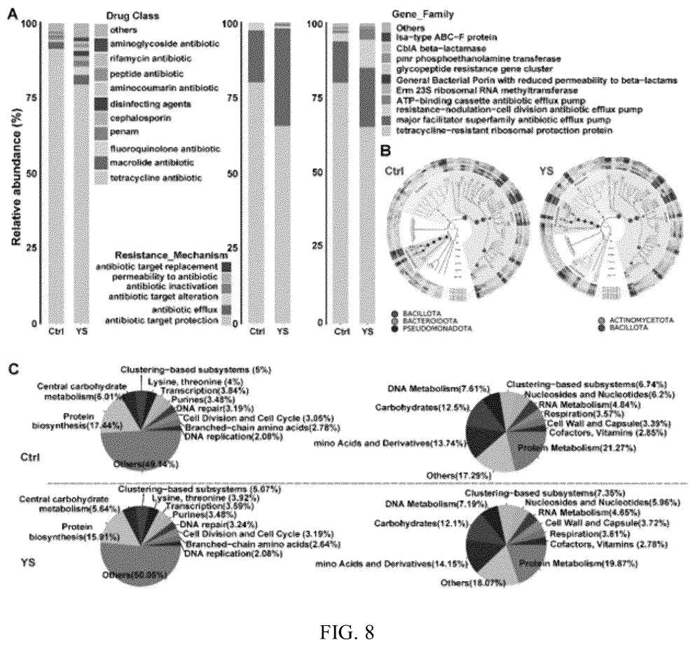

To explore whether oral administration of YS has an impact on the microecology of intestinal flora, mice were treated with probiotic YS by oral gavage for 34 consecutive days (once every 2 days), and intestinal contents were collected for metagenomic sequencing analysis. The specific results are shown in FIG. 8.

In FIG. 8, A is the effect of YS on Drug Class, Resistance Mechanism, and Gene Family in intestinal flora, B is the effect of YS on the classification of intestinal flora, and C is the effect of YS on amino acid metabolism, protein synthesis, and DNA replication of flora (n=8). Compared with the control group PBS group, the YS treatment groups (YS-L group, YS-M group, and YS-H group) can significantly regulate the metabolism of intestinal flora and bacterial gene expression; through cluster analysis of flora expression, it is found that YS has an impact on the classification of intestinal flora; and YS has an impact on flora metabolism, protein expression, DNA replication, and so on.

The preliminary experimental results show that YS can regulate the microecology of intestinal flora and may affect the body's immunity by regulating intestinal flora.

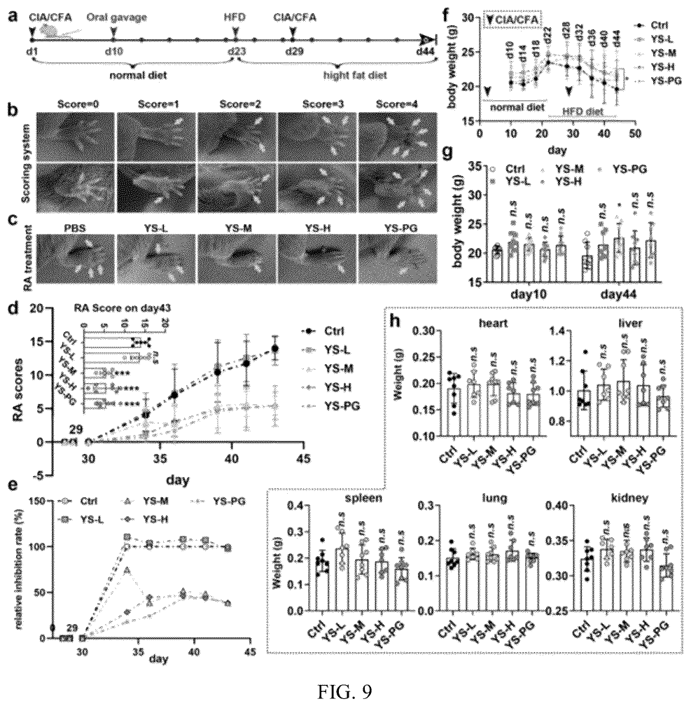

Drug induction was performed on 8-week-old male DBA/1 mice using a booster immunization regimen of subcutaneous injection at the base of the mouse tail. The steps were as follows: Day 1: Subcutaneous injection of mycobacterium type II collagen containing CFA (1 mg/mL); Day 15/25/30/34/36/39/41/43: the mouse joints were scored respectively; Day 23: Subcutaneous booster injection of type II collagen emulsion containing CFA (5 mg/ml mycobacteria); Day 10-44: YS treatment was given every 3 days. The amelioration of RA symptoms after oral administration of yeast shells and the drug safety were investigated. The specific RA model construction and related tests are shown in FIG. 9.

In FIG. 9, a is the construction process of the RA mouse model, b is the standard chart of RA mouse joint scoring, c is the chart of RA mouse joint symptoms after different treatments, d is the statistical results of the scores of RA mice in different treatment groups, e is the relative cure effect of RA mice after different treatments, and f is the weight change of mice from day 1 to day 44, g is the weight change of RA mice before and after treatment, h is the weight statistics of the main organs of mice. According to the RA scoring criteria, RA mice given different doses of YS had different changes in symptoms. The high-dose YS group effectively ameliorated the joint swelling symptoms and joint scores of RA mice, and the high-dose YS administration did not affect the weight of mice, and the high-dose YS had the same therapeutic effect as the positive control group YS-PG. By collecting the main organs of mice, it was found that YS had no effect on the weight of mouse tissues and organs, indicating that YS has a certain safety.

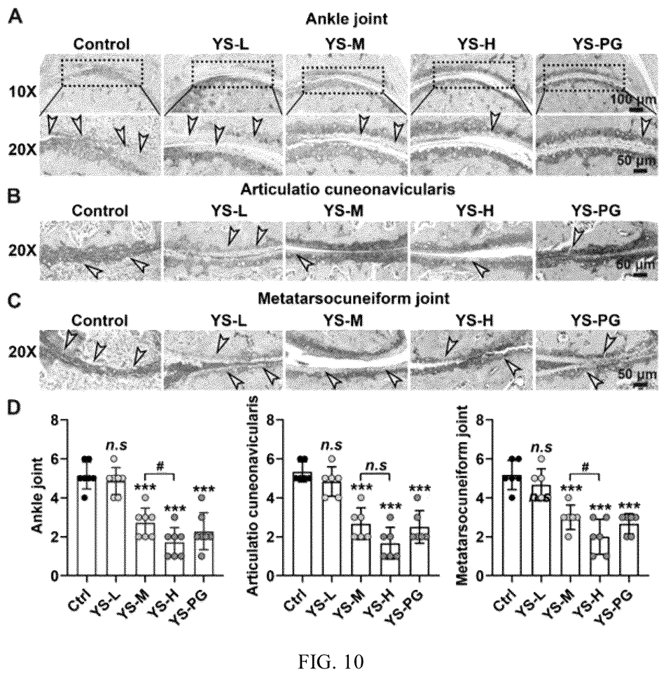

The mouse paws were collected for safranin-fixing green staining, and the results are shown in FIG. 10.

In FIG. 10, A is the result of safranin-fixing green staining of the ankle joint, B is the result of safranin-fixing green staining of the articulatio cuneonavicularis, C is the result of safranin-fixing green staining of the metatarsocuneiform joint, and D is the score of the ankle joint, articulatio cuneonavicularis, and metatarsocuneiform joint of the mouse paw. Compared with the control group, both medium and high-doses of YS can effectively improve cartilage repair in the ankle joint, articulatio cuneonavicularis, and metatarsocuneiform joint, and the results of safranin-fixing green staining showed that the high-dose group of YS-H had a better therapeutic effect on RA mice.

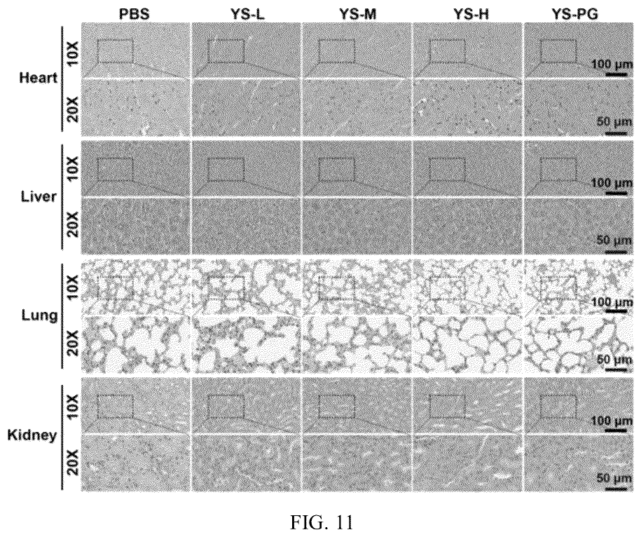

Mouse heart, liver, lung and kidney tissues were collected for H&E histological staining. The results are shown in FIG. 11.

As can be seen from the data in FIG. 11, compared with the control group, neither the medium nor high doses of YS significantly changed the cell morphology in the tissue, indicating that oral YS does not produce histological toxicity to mice and has good biosafety.

The above content is only to illustrate the technical idea of the present disclosure, and cannot limit the scope of protection of the present disclosure. Any changes made on the basis of the technical solution in accordance with the technical idea proposed by the present disclosure shall fall within the scope of protection of the claims of the present disclosure.

What is claimed is:

1. A new application of yeast shell in the preparation of drugs for treating immune diseases related to macrophage immunomodulation, wherein the yeast shells are directly used as drugs, the drugs are oral drugs, and the yeast shells are derived from yeast.

2. The new application according to claim 1, wherein the dosage form of the drug is selected from a group consisting of tablet, pill, powder, suspension, gel, emulsion, cream, granule, nanoparticle, capsule, suppository, injection, spray, and ampoule.

3. The new application according to claim 1, wherein the drug is a drug for preventing and/or treating immune diseases related to macrophage immunomodulation.

4. The new application according to claim 3, wherein the drug is a drug for preventing and/or treating rheumatoid arthritis (RA).

5. The new application according to claim 4, wherein RA comprises clinically early, established or advanced RA.

6. The new application according to claim 5, wherein the symptoms of RA comprise any one or more of humoral immune imbalance, imbalance between the body's oxidation and antioxidant capacity, increased inflammatory cytokines, joint swelling, joint stiffness, joint pain, bone injury, bone deformity, rheumatoid nodules and articular cartilage destruction.

7. An oral drug for preventing and/or treating immune diseases related to macrophage immunomodulation according to the new application of claim 1, wherein the oral drug comprises yeast shells.

8. The oral drug for preventing and/or treating immune diseases related to macrophage immunomodulation according to claim 7, wherein the oral drug further comprises one or more pharmaceutically acceptable excipients.

9. The oral drug for preventing and/or treating immune diseases related to macrophage immunomodulation according to claim 8, wherein the pharmaceutically acceptable excipient is at least one selected from a group consisting of pharmaceutically acceptable solvents, solubilizers, cosolvents, adhesives, emulsifiers, colorants, disintegrants, fillers, lubricants, wetting agents, osmotic pressure regulators, stabilizers, glidants, preservatives, flavoring agents, suspending agents, coating materials, aromatics, anti-adhesives, pH regulators, integrators, penetration enhancers, buffers, plasticizers, surfactants, moisturizers, thickeners, inclusion agents, absorbents, release blockers, diluents, flocculants and deflocculants, filter aids, polymer framework materials and film-forming materials.

10. A method for preparing the oral drug for preventing and/or treating immune diseases related to macrophage immunomodulation according to claim 7, comprising preparing yeast shells together with pharmaceutically acceptable excipients, wherein the method for preparing yeast shells is as follows: dissolving yeast in water, adjusting the pH value to 11-13 with alkali and then performing alkali treatment; centrifuging, resuspending the resulting precipitate in water, adjusting the pH value to 3-5 with acid and then performing acid treatment; centrifuging, collecting and washing the precipitate, and air-drying it at room temperature to obtain yeast shells.
